(19) United States
(12) Patent Application Publication
(10) Pub. No.: US 2024/0209388 A1
(43) Pub. Date: Jun. 27, 2024

(54) EXPRESSION VECTOR OF GLYPHOSATE-RESISTANT GENES GR79 AND GAT, HIGH GLYPHOSATE-RESISTANT CORN, AND DETECTION METHOD THEREFOR

(71) Applicant: BIOTECHNOLOGY RESEARCH INSTITUTE, CHINESE ACADEMY OF AGRICULTURAL SCIENCES, Beijing (CN)

(72) Inventors: Zhihong LANG, Beijing (CN); Min LIN, Beijing (CN); Shengyan LI, Beijing (CN); Wei LU, Beijing (CN); Xiangyin LI, Beijing (CN); Pengcheng LI, Beijing (CN); Mingjun ZHANG, Beijing (CN)

(73) Assignee: BIOTECHNOLOGY RESEARCH INSTITUTE, CHINESE ACADEMY OF AGRICULTURAL SCIENCES, Beijing (CN)

(21) Appl. No.: 18/565,933

(22) PCT Filed: Aug. 5, 2021
(86) PCT No.: PCT/CN2021/110762

§ 371 Date: Nov. 30, 2023

(30) Foreign Application Priority Data
Apr. 8, 2021 (CN) 202110376902.5
Jun. 7, 2021 (CN) 202110632185.8

(51) Int. Cl. C12N 15/82; C12Q 1/6895

(52) U.S. Cl. CPC C12N 15/8275; C12N 15/8205; C12Q 1/6895; C12Q 2600/13

(57) ABSTRACT

Provided are an expression vector of glyphosate-resistant genes GR79 and GAT, a high glyphosate-resistant corn, and a detection method therefor. The codons of the GR79 gene and GAT gene which have high tolerance to glyphosate are optimized and new DNA sequences are synthesized; meanwhile, a dual-gene plant expression vector pCGG is constructed. Results show that a transgenic corn transformed with the plant expression vector has high resistance to target herbicide glyphosate. The transgenic corn GG2 is a transformation event having a significant glyphosate tolerance effect, and a left border flanking sequence and a right border flanking sequence thereof are obtained by means of a chromosome walking method. The border sequences at two ends can be used as specific detection sequences for the present transformation event. Primers designed on the basis of the two border sequences can specifically detect the transgenic event GG2 and be applied to the development of a detection kit.

### TECHNICAL FIELD

The present invention belongs to the technical field of biology, and particularly relates to an expression vector of two glyphosate-resistant genes GR79 and GAT, a high glyphosate-resistant corn, and detection therefor.

### BACKGROUND

Corn (Zea mays L.) is a kind of bulk food crop, and also an important feed and industrial raw material. Weed is not only competitive with corn in aspects such as water, fertilizer, light and space, but also is prone to breed plant diseases and insect pests, which thus seriously affects growth and development of crops, leading to the decrease of crop yield and decline in quality. Therefore, weed control is an important link in the production of corn. The major weeds on the farmland of China include more than 250 varieties and cover an area of more than 40 million hectares. More than 10 million hectares of farmland are damaged seriously, leading to about 13% of crop losses caused by weeds per year on average; the direct economic loss accounts for 10%-20% of the total output value of crops. Manual weeding will cost a lot of labor force, resulting in the increase of planting costs; weeding by weeding-and-cultivating machinery is more expensive; and easy to cause soil erosion and soil hardening. Therefore, weed control by means of herbicides has been become an indispensable part of modern agriculture. However, due to the non-selective property, herbicide will inevitably affect the growth and development of crops during its use process. Since 1980s, with the rapid development of biological genetic engineering and the molecular-level research progress on the herbicide resistance of plants, it has made crops obtaining a herbicide-tolerant character possible via the transgenic technology. In 1983, the first herbicide-resistant crop tobacco came out, marking that the research of this field has led to success from exploration. Up to now, various kinds of transgenic herbicide-tolerant crops such as corn, soybean, cotton, and canola have been planted commercially, which brings huge social and economic benefits, and produces huge promoting effect on improving cultivated land utilization efficiency and labor efficiency.

Glyphosate is a kind of broad-spectrum and systemic herbicide. With its advantages of broad spectrum, efficiency, and eco-friendly property, it has become the most widely used variety of pesticide in the world. Shikimic acid pathway is an important way to the synthesis of aromatic amino acids in plants and microorganism. In the biosynthesis of aromatic amino acids in higher plants, e.g., phenylalanine, tyrosine, and tryptophan, 5-enolpyruvyl shikimate 3-phosphate synthase (EPSPS) is one of the central enzymes. EPSPS catalyzes the condensation of phosphoenolpyruvic acid (PEP) with shikimic acid-3-phosphate (S3P) during the metabolic pathway of shikimic acid. Glyphosate is a competitive analogue of PEP, and its mechanism of action is to form a stable complex EPSPS-S3P-glyphosate together with EPSPS and S3P. That is, its mechanism of action competitively inhibits the activity of EPSPS and blocks the transformation of S3P into 5-enolpyruvyl shikimate 3-phosphate synthase, which hinders the formation of compounds of aromatic amino acids, and leads to the accumulation of a large number of shikimic acid. In this way, flavonoids, phenolic compounds and other hormones and crucial metabolites are imbalanced, disrupting normal nitrogen metabolism of organism, thus leading to the death of organism. Based on the mechanism of action of herbicide glyphosate, there are three ways to cultivate glyphosate-resistant transgenic crops at present: (1) overexpression of the EPSPS protein to compensate the loss of EPSPS protein caused by glyphosate; (2) transformation of genes such as epsps/aroA to produce the EPSPS protein not sensitive to glyphosate, such as, transgenic corn NK603 and transgenic soybean 40-3-2 developed by Monsanto by the EPSPS gene of an Agrobacterium strain cp4; (3) transformation of glyphosate-N-acetyl transferase (GAT) to directly inactivate glyphosate.

The research and development of the herbicide-tolerant transgenic corn have been started earlier and developed very fast internationally. In 2017, the global transgenic corn had a planting area of 59.7 million hectares, of which the planting area of the herbicide-tolerant corn accounted for more than 90%. Currently, the varieties of commercial herbicide-tolerant transgenic corn are developed by the major international companies in seed industry, such as, the “NK603” herbicide-tolerant corn (containing a gene cp4-epsps) and the “GA21” herbicide-tolerant corn (containing a gene mepsps) developed by Monsanto; the “TC1507” insect-resistant and herbicide-tolerant corn (containing genes crylFa2 and pat) developed by Dow-DuPont; the “Bt11” insect-resistant and herbicide-tolerant corn (containing genes crylAband pat), and the “Bt176” insect-resistant and herbicide-tolerant corn (containing genes crylAb and bar) developed by Syngenta. From 2019 to 2020, China has issued the safety certificates for the production application of the “DBN9936” insect-resistant and herbicide-tolerant corn (containing genes crylAb and epsps), the “DBN9858” herbicide-tolerant corn (containing genes epsps and pat)developed by DBN Biotech as well as the “Ruifeng 125” insect-resistant and herbicide-tolerant corn (containing genes crylAb/cry2Aj and g10evo-epsps)jointed developed by RFGENE and Zhejiang University. It has great invigorating and promoting effects on the research and development of transgenic corn in China.

Genes GR79 and GAT are isolated and clonedfrom a metagenome of soil bacteria severely contaminated by glyphosate. The gene GR79 encodes EPSPS enzyme. Glyphosate cannot stop the GR79-EPSPS synthase from catalyzing PEP and S3P to generate EPSP such that aromatic amino acids and other compounds in plants can be continuously synthesized and metabolized; and plants obtain herbicide resistance, thus achieving normal growth. The gene GAT encodes glyphosate-N-acetyl transferase. Glyphosate-N-acetyl transferase provides a brand new mechanism of action which is different from GR79-EPSPS pathway for plants in glyphosate resistance. Under the action of glyphosate-N-acetyl transferase, glyphosate is acetylated by acetyl-CoA, as an acetyl donor, and secondary amine of glyphosate molecules, as an acetyl receptor such that glyphosate loses the activity of herbicide and transgenic plants obtain herbicide resistance, thereby protecting normal development and growth of plants.

Genes GR79 and GAT are candidate genes to create herbicide-resistantplants. A promoter CaMV35S was connected in front of the genes GR79 and GAT by researchers respectively to construct a plant expression vector pGBIGRGAT; and then the plant expression vector was transformed into cotton to obtain transgenic cotton. Tolerance of the transgenic cotton to glyphosate was detected in the T1 generation; when a solution of glyphosate isopropylamine was diluted to 1/200 (absolute concentration: 0.205 g/100 ml of glyphosate isopropylamine) and sprayed in a volume of 450 L/hectare, the survival rate was more than 84% (CN 103981199 B); moreover, leaves of the transgenic cotton showed low residue of glyphosate (Liang et al. Co-expression of GR79 EPSPS and GAT yields herbicideresistant cotton with low glyphosate residues. Plant Biotechnology Journal, 2017, 15:1622-1629).

Corn is an important crop. Currently, single-gene EPSPS enzymes are mainly selected and used for transgenic glyphosate-tolerant corn. Ren et al. have linked a gene AM79 (e.g., gene GR79 having the same amino acid sequence) to a chloroplast signal peptide of a small subunit of a pea RuBP carboxylase using a maize ubiquitin promoter, to construct a single-gene vector for maize transformation; and the transgenic maize could tolerate times the recommended dose (active ingredient: 3600 g/hectare) (Ren et al. Overexpression of a modified AM79 aroA gene in transgenic maize confers high tolerance to glyphosate. Journal of Integrative Agriculture, 2015, 14(3): 414-422). In the cultivation of high glyphosate-resistant corn, glyphosate-tolerant genes having different functions are particularly selected. A promoter highly expressed in monocotyledonous plants is utilized to improve the expression quantity of the exogenous gene, which can obtain the transgenic corn with strong field glyphosate tolerance, reduce the labor costs of manual weeding in the field, and thus, is more suitable for mechanized planting of corn.

The position of exogenous genes integrated into corn genome will affect the expression of the exogenous genes. If it is desired to obtain a transgenic plant having high expression quantity and good herbicide tolerance, a herbicide-tolerant corn having a prospect of industrialization can be obtained by screening from a large number of transformation events and detecting genetic stability of multiple generations. Meanwhile, the border sequence of the transgenic corn event inserted into corn genome can be used as an identity tag of the transgenic material, and an insertion site on chromosome can be regarded as an independent transformation event, and can be detected via specific primers. The expression vector of two glyphosate-resistant genes GR79 and GAT is utilized and transformed into corn to obtain a high glyphosate-resistant transgenic corn, i.e., a transformant GG2. The position inserted into corn genome is different from that of other transgenic events, and the border sequence can serve as an identity tag for specificity identification.

### SUMMARY

Directed to the demands for the field above, the present invention provides combined use of the two glyphosate-resistant genes GR79 and GAT in glyphosate tolerance. Codons of the GR79 gene and GAT gene are optimized and new DNA sequences are synthesized; a plant expression vector pCGG containing genes GR79 and GAT is constructed and transformed into corn to obtain transgenic corn plants containing genes GR79 and GAT; a high glyphosate-resistant transgenic event GG2 is then screened out. Moreover, a left border flanking sequence and a right border flanking sequence thereof after the genes are inserted into corn genome are obtained by means of a chromosome walking method. The flanking sequences at both ends of the border can serve as specific detection sequences for the transformation event. Primers designed according to the two border flanking sequences can specifically detect the transformation event GG2.

An expression vector, containing two glyphosate-resistant genes GR79 and GAT, where the GR79 gene has a nucleotide sequence as shown in SEQ ID NO.3, and the GAT gene has a nucleotide sequence as shown in SEQ ID NO.4.

The expression vector is named a plant expression vector pCGG and has a skeleton vector of pCAMBIA2300.

The plant expression vector pCGG has a structure as shown in FIG. 2.

The plant expression vector pCGG has a nucleotide sequence as shown in SEQ ID NO.5.

Use of the expression vector in glyphosate tolerance of a plant is provided, where the use is to transform the plant expression vector containing the genes encoding proteins GR79 and GAT into a plant to express the GR79 protein and the GAT protein such that the plant has a character of resisting glyphosate.

A method for transformation is an Agrobacterium-mediated transformation method.

The plant is corn.

A glyphosate-resistant corn transformed with genes GR79 and GAT is provided, where the GR79 gene has a nucleotide sequence as shown in SEQ ID NO.3, and the GAT gene has a nucleotide sequence as shown in SEQ ID NO.4.

A left border flanking sequence of an exogenous insertion fragment of the glyphosate-resistant corn GG2 transformed with genes GR79 and GAT is provided, as shown in positions 269-462 of SEQ ID NO.6, or as shown in positions 1-440 of SEQ ID NO.8.

A right border flanking sequence of an exogenous insertion fragment of the glyphosate-resistant corn GG2 transformed with genes GR79 and GAT is provided, as shown in positions 456-1773 of SEQ ID NO.7, or as shown in positions 6556-7873 of SEQ ID NO.8.

A specific primer pair for PCR reaction detection designed according to the left border flanking sequence is provided.

The left border flanking sequence has a specific primer pair of:

GG2-Left-F3:
5′-GGAGCAAGGAAGCGGACTAC-3′,
GG2-Left-R1:
5′-CCCCACATCCTGATGTACAAG-3′.

A specific primer pair for PCR reaction detection designed according to the right border flanking sequence is provided.

The right border flanking sequence has a specific primer pair of:

GG2-Ubi-F1:
5′-ATGATTCTCTAAAACACTG-3′,
GG2-Right-R1:
5′-GCGAACATAGCGTCTTAC-3′.

A PCR reaction detection method of a glyphosate-resistant corn GG2 transformed with genes GR79 and GAT, where primers in the PCR reaction are the above specific primer pair.

the specific primer pair is:

GG2-Left-F3:
5′-GGAGCAAGGAAGCGGACTAC-3′,
GG2-Left-R1:
5′-CCCCACATCCTGATGTACAAG-3′.

a fragment obtained by the PCR reaction has a size of 734 bp; or the specific primer pair is:

GG2-Ubi-F1:
5′-ATGATTCTCTAAAACACTG-3′,
GG2-Right-R1:
5′-GCGAACATAGCGTCTTAC-3′.

a fragment obtained by the PCR reaction has a size of 1773 bp.

A kit for detecting a glyphosate-resistant corn is provided, including the specific primer pair of the left border flanking sequence, and/or the specific primer pair of the right border flanking sequence; where the left border flanking sequence is shown in positions 269-462 of SEQ ID NO.6, and the right border flanking sequence is shown in positions 456-1773 of SEQ ID NO.7.

The left border flanking sequence has specific primers of:

GG2-Left-F3:
5′-GGAGCAAGGAAGCGGACTAC-3′,
GG2-Left-R1:
5′-CCCCACATCCTGATGTACAAG-3′.

The right border flanking sequence has specific primers of:

GG2-Ubi-F1:
5′-ATGATTCTCTAAAACACTG-3′,
GG2-Right-R1:
5′-GCGAACATAGCGTCTTAC-3′.

Use of the above flanking sequence, the specific primers of the flanking sequence, and the kit for detecting a glyphosate-resistant corn in detecting a transgenic corn.

In the present invention, codons of the nucleotide sequences of the genes GR79 (SEQ ID NO.1) and GAT (SEQ ID NO.2) are optimized, and new genes GR79 (SEQ ID NO.3) and GAT (SEQ ID NO.4) are synthesized via artificial synthesis, so as to construct a bivalent GR79 and GAT plant expression vector, and the expression vector is transformed into corn to obtain 103 TO generation of transformation plants in total, of which 76 positive plants are detected via PCR test. All the positive transformation plants are transferred to a greenhouse and seeds thereof are harvested. T1 generation of transgenic corn is planted in the field and artificially sprayed with glyphosate (the spraying volume is 1, 2, and 4 times the medium dose on the pesticide registration tag, mixed with 450 L/hectare water; the medium dose of Roundup (trade name of glyphosate pesticide) is 900 g active ingredient/hectare, the same below) after growing to four to six-leaf stage. The glyphosate-resistant transgenic corn GG2 (accession number: CGMCC No. 20132), after multi-generation of screenings, is not endangered by glyphosate and has remarkable resistance to glyphosate. Furthermore, a left border flanking sequence and a right border flanking sequence thereof are obtained by means of a chromosome walking method. The flanking sequences at both ends can serve as specific detection sequences for the transformation event. Primers designed according to the two border flanking sequences can specifically detect the transformation event GG2, and can be applied to the development of a detection kit.

Test results of the field non-target herbicide tolerance indicate that the glyphosate-resistant corn transformed with bivalent GAT and GR79 can be killed by other types of herbicides, not leading to a “superweed”. In addition, compared with the researches at the same period, the high glyphosate herbicide-resistant transformation event obtained by screening from transgenic glyphosate-resistant corn having single expression of GAT protein has a probability obviously lower than that of the bivalent transgenic glyphosate-resistant corn. Moreover, for partial single-gene GAT high-resistant events, leaves will suffer mild symptoms of herbicide damage at the early stage after being sprayed with high concentration of glyphosate (4 times the medium dose), and there is no survival plant after spraying 8 times the medium dose of glyphosate. It indicates that the coordinated expression of genes GR79 and GAT can improve the glyphosate tolerance of corn more and thus, is of important significance to cultivate high glyphosate-resistant corn with independent intellectual property.

Transgenic corn GG2 is classified and named Zea mays.

Accession number: CGMCC No. 20132

Deposit date: Jan. 14, 2021

Deposit institution: China Committee for Culture Collection of Microorganisms (CCCCM)

Deposit address: Institute of Microbiology, Chinese Academy of Sciences, No. 3, Yard 1, West Beichen Road, Chaoyang District, Beijing, postcode: 100101

### BRIEF DESCRIPTION OF DRAWINGS

FIG. 1 is a schematic diagram of a plant expression vector pCGAT;

FIG. 2 is a schematic diagram of a plant expression vector pCGG;

FIG. 3 shows PCR detection of a GAT gene (A) in T1 generation of monovalent GAT transgenic corn, GAT gene (B) and GR79 gene (C) in bivalent GAT and GR79 transgenic corn;
where M denotes a DNA molecular weight standard, Super Marker; CK+: a product amplified with a plasmid pCGG as a template; CK-: a product amplified with genomic DNA of non-transgenic corn as a template; 0: blank, a product amplified with water as a template; 1-9: products amplified with genomic DNA of T1 generation of GG1-GG9 transgenic corn as a template;

FIG. 4 shows detection of field glyphosate tolerance of transgenic corn GG2;
where figure A shows that the BC4 generation of GG2 is sprayed with 4 times glyphosate; figure B shows that the BC5 generation of GG2 is sprayed with 4 times glyphosate;

FIG. 5 shows results after a monovalent GAT glyphosate-resistantcorn is sprayed with 4 times (A) and 8 times (B) a medium dose of glyphosate;

FIG. 6 shows a southern blot result of transgenic corn GG2;
where figure A shows a test result of a GAT probe; figure B shows a test result of a GR79 probe; Marker is a DNA molecular weight standard and consists of 7 DNA fragments; the band size is 23,130 bp, 9,416 bp, 6,557 bp, 4,361 bp, 2,322 bp, 2,027 bp and 564 bp from top to bottom, respectively; CK+: pCGG plasmid/HindIII digestion; CK-: genomic DNA of non-target transgenic corn/HindIII digestion;

FIG. 7 is a schematic diagram showing restriction enzyme cutting sites of an insertion fragment and expected size of southern blot bands;

FIG. 8 shows detection of a left border flanking sequence of the transgenic corn GG2 by chromosome walking;
M is a Trans5K DNA Marker; 1^{st} shows a result of the first round PCR; 2^{nd} shows a result of the second round PCR; and 3^{rd} shows a result of the third round PCR;

FIG. 9 shows PCR electrophoretogram for the specificity of a right border (A) and a left border (B) of transgenic corn GG2 from generations T2 to T4; Marker: Trans5K DNA Marker; CK1 (H₂O): blank control with H₂O as a template; CK2(pCGG): a product amplified with a plasmid pCGG as a template; CK3(sister event GG3): a product amplified with genomic DNA of a transformant GG3 as a template; CK4(sister event GG4): a product amplified with genomic DNA of a transformant GG4 as a template; CK5(receptor B104): a product amplified with genomic DNA of a transformant B104 as a template; CK6(Z58): a product amplified with genomic DNA of a back-crossing transformation receptor Z58 as a template; T2: a product amplified with genomic DNA of a T2 generation of transformant GG2; T3: a product amplified with genomic DNA of a T3 generation of transformant GG2; and T4: a product amplified with genomic DNA of a T4 generation of transformant GG2;

FIG. 10 shows a position of the insertion fragment of transgenic corn GG2 in corn genome;

FIG. 11 shows detection of field non-target herbicide tolerance of the bivalent GAT and GR79 glyphosate-resistant corn GG2.

### DETAILED DESCRIPTION OF EMBODIMENTS

The present invention will be further described in combination with the examples below.

The following biological materials involved are all preserved in labs of the applicant and may be open to the public.

### Example 1. Transformation and Optimization of Codons of the Genes GR79 and GAT

Gene GR79 was isolated from a metagenome of soil bacteria severely contaminated by glyphosate and cloned; and has independent intellectual property (patent number: ZL 200710177090.1). Gene GR79 has a coding sequence of 1338 bp and a nucleotide sequence as shown in SEQ ID NO.1; and EPSPS enzyme encoded thereby consists of 445 amino acids. The coding sequence of gene GR79 was optimized according to the codon preferred by plant; the original gene GR79 has a GC content of 45.85%, and the optimized gene GR79 has a GC content of 64.56%, and the optimized nucleotide sequence is shown in SEQ ID NO.3. Gene GAT was cloned by functional screening from a gene library of total DNA of soil microorganism contaminated by glyphosate; and has independent intellectual property right (patent number: ZL 200510086626.X). Gene GAT has a coding sequence of 441 bp and a nucleotide sequence as shown in SEQ ID NO.2; and glyphosate acetyltransferase encoded thereby consists of 146 amino acids. The coding sequence of gene GAT was optimized according to the codon preferred by plant; the original gene GAT has a GC content of 47.86%, and the optimized gene GAT has a GC content of 63.90%, and the optimized nucleotide sequence is shown in SEQ ID NO.4.

### Example 2. Construction of a Monovalent GAT Plant Expression Vector, and a Bivalent GAT and GR79 Plant Expression Vector

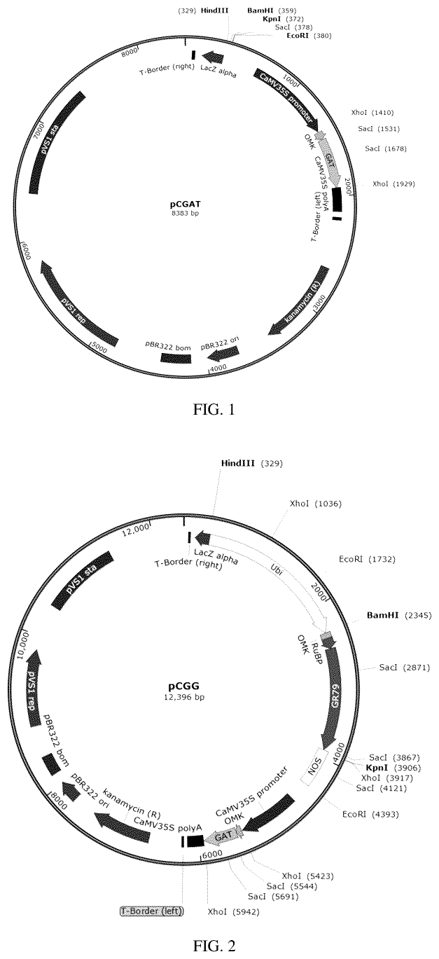

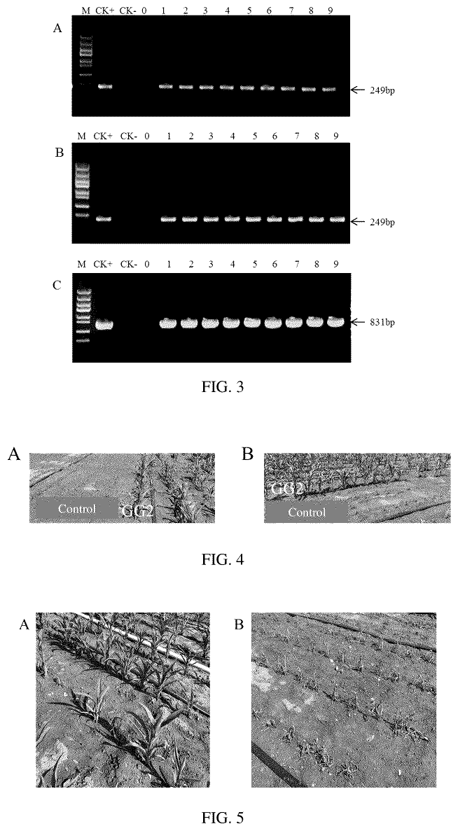

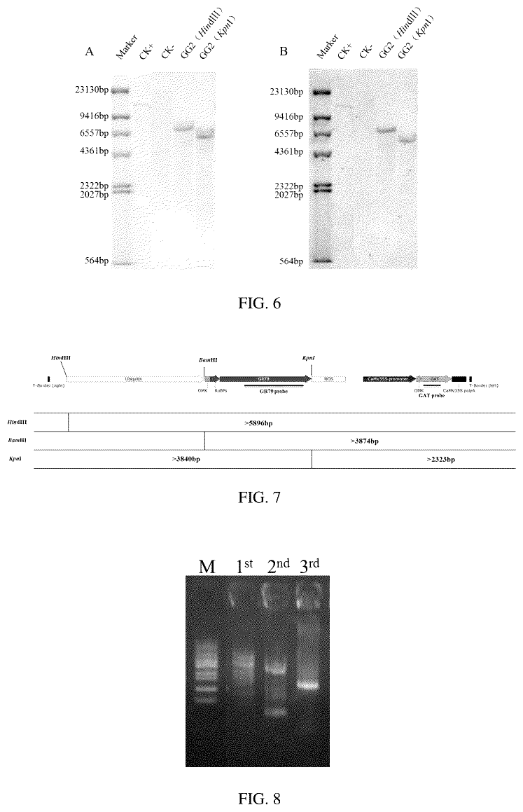

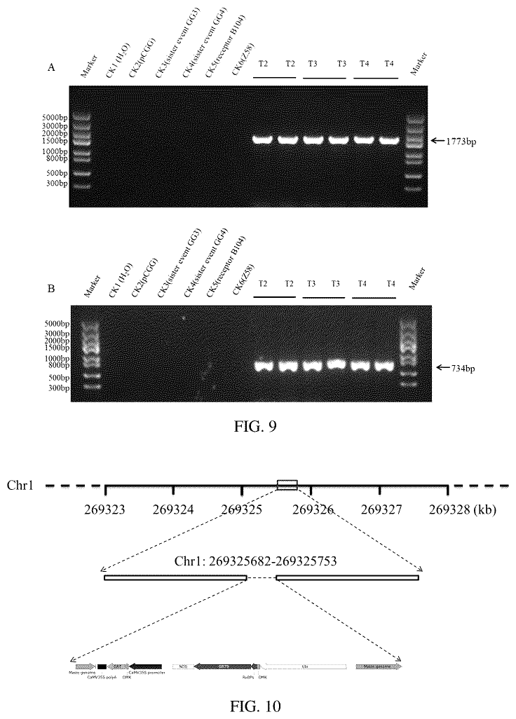

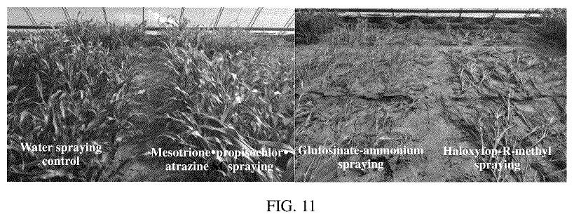

The optimized genes GAT and GR79 were artificially synthesized, and meanwhile, an OMK sequence for enhancing genetic expression was added upstream of the gene GAT; an OMK sequence for enhancing genetic expression and a signal peptide sequence ZmRuBP of corn chloroplast were added upstream of the gene GR79. A gene nptll was removed from T-DNA via Xhol digestion in a commercial vector pCAMBIA2300; the synthesized OMK-GAT fragment was linked by seamless cloning, to construct a vector, called pCGAT (as shown in FIG. 1). The vector constructed contains a single GAT gene. pUC57-UN is an intermediate vector, and contains a promoter Ubiquitin and a terminator NOS (the plasmid was preserved in the Lang Zhihong research group of Biotechnology Research Institute, CAAS, and can be open to the public). The synthesized OMK-RuBPs-GR79 fragment was linked into the vector pUC57-UN via BamHI and KpnI digestion; a GR79 expression cassette was linked into the pCGAT vector via HindIII and EcoRI digestion, so as to construct a final vector pCGG (as shown in FIG. 2). The final vector contains genes GAT and GR79.

### Example 3. Obtaining of a Monovalent GAT Transgenic Corn, and a Bivalent GAT and GR79 Transgenic Corn

In the present invention, the vector pCGAT and the vector pCGG were transformed into Agrobacterium EHA105, respectively by a freeze-thaw method, and then subjected to PCR identification. Corn immature embryos (about 1.2 mm) stripped fresh was used as a material and placed into an infection medium, 1 hr later, washed once with the infection medium, and then soaked into an Agrobacterium solution with the addition of 100 μM acetosyringone, and placed for 5 min. The immature embryos was then taken out and blotted up with sterilized filter paper, and put onto a co-culture medium and co-cultured for 3 d at 26° C. in the dark; a control was set. The immature embryos was then transferred onto a recovery medium and cultured for 10 d until callus was induced; the callus was then firstly degerminated and transferred onto a screening medium containing corresponding screening reagent, and subcultured once every other week; 6 weeks later after the screening, the resistant callus was transferred onto a regeneration medium for light exposure differentiation, green bud points started to emerge after about one week; the callus piece was cut such that green bud points were separated and transferred onto a regeneration medium for culture, which was beneficial to stem growth; when the stem stretched to 3-4 cm, the separated green bud points were transferred onto a regeneration medium for root induction; the corn plant was transferred to a small flowerpot in greenhouse for growth after growing sturdily and having a developed root system. Two weeks later after continuous culture, the transformed seedlings grew well and then were transferred to a greenhouse, and covered with a paper bag after the presence of cornsilk, and then pollinated after male inflorescence spread pollen; ears were harvested.

The media are as follows:

infection medium: N6 salt and vitamin N6 (Chu et al., Science Sinica, 1975, 18: 659-668), 1.5 mg/L 2,4-D, 0.7/L g proline, 68.4 g/L sucrose, 36 g/L glucose (pH 5.2) were filtered and sterilized, and stored at 4° C.; filtered and sterilized acetosyringone (AS) was added to the solution before use to obtain a final concentration of 100 μM;

co-culture medium: N6 salt and vitamin N6, 1.5 mg/L 2,4-D, 0.7 g/L proline, 30 g/L sucrose, 3 g/L phytagel (pH 5.8) were subjected to autoclaved sterilization, and then added with filtered and sterilized silver nitrate having a final concentration of 0.85 mg/L, 100 μM AS, and 300 mg/L cysteine;
recovery medium: N6 salt and vitamin N6, 1.5 mg/L 2,4-D, 0.7 g/L proline, 30 g/L sucrose, 0.5 g/L MES, 4 g/L phytagel (pH 5.8) were subjected to autoclaved sterilization, and then added with filtered and sterilized silver nitrate having a final concentration of 0.85 mg/L, and 200 mg/L carbenicillin;
screening medium: a screening reagent, 1 mM glyphosate, was added to the recovery medium; and
regeneration medium: MS salt and vitamin MS, 30 g/L sucrose, 100 mg/L inositol, and 3 g/L phytagel (pH 5.8) were subjected to autoclaved sterilization. The corn immature embryos were stripped fresh and had a length of 1.2 mm.

The Agrobacterium solution was admixed with 100 μM acetosyringone.

### Example 4. PCR detection of the monovalent GAT transgenic corn plant, and the bivalent GAT and GR79 transgenic corn plant

Genomic DNA of the transgenic corn plant was extracted by CTAB.

Primers were designed according to the optimized GAT and GR79 sequences; the GAT primer sequences: GAT-F1: 5′-TCGACGTGAACCCGATCAAC-3′, GAT-R1: 5′-TCTGCTCCCTGTAGCCCTCC-3′; the GR79 primer sequences: GR79-F1: 5′-TCAGCAGGGCGAGTGGA-3′, GR79-R1: 5′-TCGTCGTGCGGGTTCAG-3′. The target fragment obtained by amplifying the gene GAT had a size of 249 bp, and the target fragment obtained by amplifying the gene GR79 had a size of 831 bp;

PCR reaction system (20 μL) of the gene GAT:

| | | |
|---|---|---|
| Corn genomic DNA | 100 | ng |
| GAT-F1 (10 pmol/μL) | 0.5 | μL |
| GAT-R1 (10 pmol/μL) | 0.5 | μL |
| 2 × Taq Mixture | 10 | μL |

| | |
|---|---|
| ddH₂O | up to 20 uL |

PCR reaction conditions of the gene GAT:

95° C. 3 min; 95° C. 20 s, 58° C. 15 s, 72° C. 20 s, 30 cycles; 72° C. 5 min; 4° ° C. pause.

PCR reaction system (20 μL) of the gene GR79:

| | | |
|---|---|---|
| Corn genomic DNA | 100 | ng |
| GR79-F1 (10 pmol/μL) | 0.5 | μL |
| GR79-R1 (10 pmol/μL) | 0.5 | μL |
| 2 × Taq Mixture | 10 | μL |

| | |
|---|---|
| ddH₂O | up to 20 uL |

PCR reaction conditions of the gene GR79; 95° C. 3 min; 95° C. 20 s, 59.4° ° C.15 s, 72° ° C.40 s, 30 cycles; 72° ° C.5 min; 4° C. pause.

PCR test results are shown in FIG. 3.

During the transformation of genes GAT and GR79 into corn, 103 TO generation of transformed plants were obtained in total, including 76 double-positive GAT and GR79 transgenic corn plants detected by PCR, having a positive rate of 73.79%. Meanwhile, 46 events transformed with the single gene GAT, including 38 positive plants detected by PCR, having a positive rate of 82.6%.

### Example 5. Screening of a High Glyphosate-Resistant Event of Monovalent

GAT transgenic corn, and a high glyphosate-resistant event of bivalent GAT and GR79 transgenic corn

T1 generation of materials of the 76 double-positive GAT and GR79 transformation events detected by PCR were sown in the field, sprayed with glyphosate at the four-leaf stage in a medium dose (active ingredient: 900 g/hectare) on the pesticide registration tag; there were 72 survival transformation events and the rate of zero symptom of pesticide damage was 94.7%. 2 times the medium dose of glyphosate (active ingredient: 1800 g/hectare) was continuously applied, and survey was conducted 4 weeks later after application, 63 transformation events were free of symptom of pesticide damage. The T2 generation of materials of the screened 63 transformation events were sown and sprayed with glyphosate at the four to five-leaf stage in a spraying amount of 1-fold, 2 times, and 4 times the medium dose (active ingredient: 900 g/hectare) on the pesticide registration tag. The rate of survival and symptom of pesticide damage of each transformation event were surveyed and recorded 4 weeks later after application. 47 transformation events were free of symptom of pesticide damage after sprayed with 4 times the medium dose of glyphosate and thus, were high glyphosate-resistant materials, accounting for 61.84% of the 76 positive transgenic materials. The screening concentration continued to be increased; when it was up to 8 times (active ingredient: 7200 g/hectare) the medium dose, 6 transformation events were not affected. The GG2 transformation event was not endangered by glyphosate during the multi-generation of screening process and had remarkable glyphosate tolerance (FIG. 4).

To determine the effect of the combined use of double genes on high glyphosate tolerance, the transgenic corn transformed with the single gene GAT was subjected to glyphosate tolerance test according to the same screening process, 8 transformation events which could be resistant to 4 times glyphosate were obtained finally, accounting for 21.05% of the 38 positive transgenic materials. By comparison of the two, the bivalent GAT and GR79 transgenic glyphosate-resistant corn was easier to obtain high glyphosate-resistant materials. In addition, compared with the transformation event of the high glyphosate-resistant corn transformed with double genes GAT and GR79, even though the transformation event of the high glyphosate-resistant corn transformed with the gene GAT could be resistant to 4 times glyphosate, in partial events, leaves thereof would suffer mild symptoms of pesticide damage (A in FIG. 5) in a few days after being sprayed with 4 times the medium dose of glyphosate. There was no survival plant (B in FIG. 5) after the GAT transgenic corn was sprayed with 8 times (active ingredient: 7200 g/hectare) the medium dose of glyphosate.

### Example 6. Identification of Field Glyphosate Tolerance of the Transgenic Corn Event GG2

Field herbicide tolerance of transgenic corn was tested in accordance with the Test for Environmental Safety of Transgenic Plant and its Products Part 1 of Herbicide-Resistant Corn: herbicide Tolerance (No. 953 bulletin-11.1-2007 of the Ministry of Agriculture and Rural Affairs).

The test material was the transgenic corn GG2, and the corresponding non-transgenic corn. The herbicide was glyphosate. Block design (not random) was conducted with 2 repeats; the block had an area of 30 m² (5 mx6 m), line space of 60 cm, and row space of 25 cm. An isolation belt (a width of 1.0 m) was set among the blocks. Treatments include: transgenic corn not sprayed with a herbicide, transgenic corn sprayed with a target herbicide (glyphosate), non-transgenic corn not sprayed with a herbicide, and non-transgenic corn sprayed with a target herbicide (glyphosate). The application amount of the herbicide was 1-fold, 2 times, and 4 times the medium dose (active ingredient: 900 g/hectare) on the pesticide registration tag, respectively. The herbicide was applied at the four to five-leaf stage of corn. The rate of survival, plant height, and symptom of pesticide damage were surveyed and recorded 1, 2, and 4 weeks later after application, respectively. The symptom of pesticide damage was graded according to GB/T 19780.42. Glyphosate in the transgenic corn GG2, and in its corresponding transgenic control, and non-transgenic control was identified. The identification results of the field glyphosate tolerance of the consecutive two generations show that: compared with the transgenic corn not sprayed with a herbicide, the transgenic corn GG2 was free of symptom of damage at each stage and had no significant difference in plant height (Table 1 and Table 2) after being sprayed with different treatments of glyphosate. This indicates that the transgenic corn GG2 could be resistant to 4 times the medium dose of glyphosate and below, and achieve the stable inheritance of the glyphosate tolerance character.

TABLE 1
Survey on the plant height of the transgenic corn GG2 treated with glyphosate

| | | 1 week (cm) | | 2 weeks (cm) | | 4 weeks (cm) | |
|---|---|---|---|---|---|---|---|
| Material | Treatment | BC4 | BC5 | BC4 | BC5 | BC4 | BC5 |
| Zheng | Not | 46.04 ± | 29.79 ± | 71.09 ± | 51.69 ± | 136.5 ± | 116.06 ± |
| 58 | sprayed | 4.58b | 3.6a | 4.98b | 6.46a | 3.67b | 6.86a |
| GG2 | Not | 58.48 ± | 34.95 ± | 88.31 ± | 54.45 ± | 160.7 ± | 137.14 ± |
| | sprayed | 1.09a | 1.88b | 1.53a | 5.55a | 5.41a | 8.07b |
| GG2 | 1-fold the | 58.52 ± | 33.87 ± | 85.59 ± | 51.71 ± | 158.39 ± | 136.37 ± |
| | medium dose | 3.19a | 2.03b | 3.66a | 4.51a | 2.99a | 5.23b |
| GG2 | 2 times the | 51.24 ± | 33.11 ± | 79.38 ± | 54.29 ± | 147.75 ± | 139.65 ± |
| | medium dose | 2.63ab | 2.02b | 3.78ab | 3.27a | 5.06ab | 6.71b |
| GG2 | 4 times the | 59.81 ± | 34.65 ± | 88.41 ± | 53.78 ± | 158.53 ± | 137.39 ± |
| | medium dose | 1.59a | 2.8b | 2.34a | 4.6a | 1.79a | 7.2b |

Note:
data in the table denote a mean value ± a standard deviation; different English lowercases behind of the same column of data denote a significant difference (P < 0.05) between different treatments. The same below.

TABLE 2
Survey on the damage percent of GG2 treated with glyphosate

| | | 1 week (cm) | | 2 weeks (cm) | | 4 weeks (cm) | |
|---|---|---|---|---|---|---|---|
| Material | Treatment | BC4 | BC5 | BC4 | BC5 | BC4 | BC5 |
| Zheng | Not | 0.0 ± | 0.0 ± | 0.0 ± | 0.0 ± | 0.0 ± | 0.0 ± |
| 58 | sprayed | 0.0a | 0.0a | 0.0a | 0.0a | 0.0a | 0.0a |
| GG2 | Not | 0.0 ± | 0.0 ± | 0.0 ± | 0.0 ± | 0.0 ± | 0.0 ± |
| | sprayed | 0.0a | 0.0a | 0.0a | 0.0a | 0.0a | 0.0a |
| GG2 | 1-fold the | 0.0 ± | 0.0 ± | 0.0 ± | 0.0 ± | 0.0 ± | 0.0 ± |
| | medium dose | 0.0a | 0.0a | 0.0a | 0.0a | 0.0a | 0.0a |
| GG2 | 2 times the | 0.0 ± | 0.0 ± | 0.0 ± | 0.0 ± | 0.0 ± | 0.0 ± |
| | medium dose | 0.0a | 0.0a | 0.0a | 0.0a | 0.0a | 0.0a |
| GG2 | 4 times the | 0.0 ± | 0.0 ± | 0.0 ± | 0.0 ± | 0.0 ± | 0.0 ± |
| | medium dose | 0.0a | 0.0a | 0.0a | 0.0a | 0.0a | 0.0a |

### Example 7. Analysis on the Insertion Site of the Exogenous Genes of the Transgenic Corn Event GG2

1. Bulk extraction of the corn genomic DNA (slightly improved CTAB method):
(1) 5 g leaves were weighed and fully ground to powder under liquid nitrogen (the material should be not molten), and then added to a 50 mL of centrifugal tube;
(2) 15 mL of 2×CTAB buffer solution (Tris 100 mM, 1.4 M NaCl, 20 mM EDTA, 2% CTAB, 0.1% mercaptoethanol) was added and mixed well, the centrifugal tube was then treated by water bath for 1 hr at 65° C.;
(3) after cooling to room temperature, 15 mL chloroform/isoamyl alcohol (24:1) was added, turned upside down and mixed well to an emulsion, and then placed at room temperature for 15-60 min;
(4) the emulsion was centrifuged for 15 min at 12,000 rpm and room temperature;
(5) supernatant was transferred into a clean centrifugal tube, and admixed with isopropyl alcohol in ⅔ of volume, turned upside down several times; DNA was picked up and put to a 1.5 mL of clean centrifugal tube;
(6) the DNA was washed with 70% ethanol twice, and dried in the air for 1 hr;
(7) after the DNA was dissolved by 500 μL TE, 5 μL RNase A (10 mg/mL) was then added, and dissolved at 4° C. or dissolved at 37ºC for 1 hr over the night;
(8) 500 μL phenol was added; the mixed solution was turned upside down and mixed well, and centrifuged for 5 min at 12,000 rpm, and supernatant was transferred into another centrifugal tube;
(9) 250 μL phenol and 250 μL chloroform were added, respectively; the mixed solution was turned upside down and mixed well, and centrifuged for 5 min at 12,000 rpm, and supernatant was transferred into another centrifugal tube;
(10) 500 μL chloroform was added; the mixed solution was turned upside down and mixed well, and centrifuged for 5 min at 12,000 rpm, and supernatant was transferred into a 10 mL centrifugal tube;
(11) TE was added until 3 mL; 2 times volume of absolute ethyl alcohol and 1/10 times volume of 3 M NaAc were then added; the mixed solution was turned upside down several times;
(12) the precipitated DNA was cleaned twice with 70% ethanol, and transferred to a 1.5 mL centrifugal tube, dried in the air and dissolved into 500 μL TE; DNA was quantified for further use.

2. Preliminary experiment of a small amount of enzyme digestion of the genomic DNA

| | |
|---|---|
| Genomic DNA | 1 μg |
| Enzyme (10 U/μL) | 1 μL |
| 10 × Buffer | 2 μL |
| Total | 20 μL |

The above materials were mixed well and subjected to enzyme digestion reaction for 2-3 hrs at 37° C.; the enzyme digestion reactant was subjected to electrophoretic separation via 0.7% agarose, and the enzyme digestion effect was examined.

3. A large amount of enzyme digestion of the genomic DNA:

| | | |
|---|---|---|
| Genomic DNA | 100 | μg |
| Enzyme (10 U/μL) | 5 | μL |
| (Hind III and Kpn I | | |
| were selected in this | | |
| experiment for | | |
| enzyme digestion) | | |
| 10 × Buffer | 40 | μL |
| Total | 400 | μL |

The above materials were mixed well, and then subjected to enzyme digestion reaction for 10 hrs at 37° C.; 2 μL enzyme-digested product was subjected to electrophoretic separation; the enzyme digestion effect was examined. After the completion of the enzyme digestion, the enzyme-digested product was precipitated, and admixed with 1/10 times volume of 3 M NaAc, 2 times volume of absolute ethyl alcohol (precooled at −20° C.), and mixed well and placed at −20° ° C. for 2 hrs; the mixed solution was centrifuged for 20 min at 4° C. and 12,000 rpm; supernatant was discarded; precipitate was added with 1 mL 70% ethanol and centrifuged for 2 min at 12,000 rpm, and supernatant was discarded; precipitate was blown-dried and dissolved into 30 μL ddH₂O for further use.

4. Probe preparation

A probe was prepared according to the instructions of PCR DIG Probe Synthesis Kit.

Primers of the GAT gene probe:

GAT probe-F1:
5′-TCGACGTGAACCCGATCAAC-3′,
GAT probe-R1:
5′-TCTGCTCCCTGTAGCCCTCC-3′,

The GAT gene probe had a size of 249 bp.

Primers of the GR79 gene probe:

GR79 probe-F1:
5′ TCAGCAGGGCGAGTGGA 3′,
GR79 probe-R1:
5′ TCGTCGTGCGGGTTCAG 3′,

The GR79 gene probe had a size of 831 bp.

PCR reaction system was as follows:

| | | |
|---|---|---|
| pCGG plasmid | 10-100 | pg |
| probe-F1 | 5 | μL |
| probe-R1 | 5 | μL |
| PCR DIG probe | 5 | μL |
| synthesis mix | | |
| PCR buffer | 5 | μL |
| Enzyme mix | 0.75 | μL |

| | |
|---|---|
| ddH₂O | add up to 50 μL |

PCR reaction conditions were as follows:

| Temperature | Time | Cycle | |
|---|---|---|---|
| 95° C. | 2 min | 1 | |
| 95° C. | 30 s | | |
| 56° C. | 30 s | {close oversize brace} | 10 |
| 72° C. | 40 s | | |
| 95° C. | 30 s | | |
| 56° C. | 30 s | | 20 |
| 72° C. | 40 s + | | |
| | 20 s | {close oversize brace} | |
| | each | | |
| | cycles | | |

| | | |
|---|---|---|
| 72° C. | 7 min | 1 |

At the end of PCR, the DIG-labeled probe was detected by electrophoresis and a concentration was determined.

5. Southern blot
(1) 0.7% agarose gel was prepared; 6 μL 6×loading buffer was added to 30 μL of enzyme-digested product of genomic DNA and subjected to electrophoretic separation, and then subjected standing for 10 min after sample loading; a low voltage was applied in the beginning, the voltage was increased to 50 V when bromophenol blue ran out of the sampling well at 2-3 cm, electrophoresis was conducted for 5-6 h;
(2) after the completion of electrophoresis, the gel was treated as follows, respectively: the gel was soaked into 0.125 M hydrochloric acid for 10 min; bromophenol blue in the gel turned yellow; the gel was treated with distilled water for 5 min, and then soaked into a neutralization solution for 30 min;
(3) DNA was transferred onto a nylon membrane by a capillary transfer method (specific operation is referring to the Lab Manual of Molecular Cloning);
(4) after DNA was transferred onto the nylon membrane, the nylon membrane was soaked by 6×SSC for 5 min, and then placed onto a super clean bench, blown-dried or dried in the air at room temperature;
(5) the nylon membrane was dried for 2 hrs at 80° C.; a DNA sample was immobilized;
(6) preparation of a DIG-labeled probe: GAT and GR79-labeled probe was prepared according to the probe preparation method of the PCR DIG Probe Synthesis Kit (purchased from Roche), and the probe concentration was determined;
(7) prehybridization: the nylon membrane was put to a hybridization tube with forceps carefully and bubble should be avoided, 10 mL of DIG Easy Hyb hybridization solution (Digoxin-labeled detection kit II were purchased from Roche) preheated at 42° ° C. was added and pre-hybridized for 3 hrs at 42° C.;
(8) hybridization: the probe was treated first, and the labeled probe was degenerated for 6 min at 99° ° C., and immediately placed into ice and cooled for 2 min. 7 mL of the DIG Easy Hyb hybridization solution was taken and added with the treated probe (25 ng/ml Hyb hybridization solution), slightly mixed well, bubbles should be avoided, then placed into a hybridization oven and hybridized for 16-20 hrs at 42° ° C.;
(9) membrane washing: the membrane was washed twice with 50 mL of 2×SSC, 0.1% SDS solution at room temperature first, for 15 min each time; the membrane was then washed twice with 50 mL of 0.5×SSC, 0.1% SDS solution at 65° C., for 30 min each time; the membrane was carefully taken out and transferred into a petri dish containing 50 mL Washing buffer with forceps, oscillated and washed for 1-5 min;
(10) the membrane was incubated with 100 ml 1×Blocking solution for 60 min at room temperature;
(11) the membrane was then incubated with 20 ml Antibody solution for 30 min;
(12) washed twice with 50 ml of Washing buffer, for 30 min each time; (13) the membrane was balanced for 2-5 min in 20 ml of Detection buffer;
(14) the membrane was laid between two layers of a preservative film with forceps; the upper layer of preservative film was lifted first, and 1 ml of CSPD substrate was added therein; the upper layer of preservative film was slowly put down from one end such the substrate was uniformly covered on the surface of the membrane, then standing for 5 min at room temperature;

(15) excess liquid was removed with a glass rod, and the substrate outside the membrane was blotted up with filter paper, and the membrane was incubated for 10 min at 37° ° C.;

(16) image analysis was conducted with an AI600 (GE, US) digital chemiluminescence imaging analysis system. Results are shown in FIG. 6.

Analysis was conducted on the basis of the Southern blot results of the transgenic corn event GG2: there was an HindIII enzyme cutting site and a Kpn I enzyme cutting site in the T-DNA sequence inserted into the corn genome. These two enzyme cutting sites will not affect the identification of copies of the genes GAT and GR79. The schematic diagram of the restriction enzyme cutting sites of the insertion fragment and expected sizes of southern blot bands are shown in FIG. 7. The GAT gene fragment and the GR79 gene fragment served as probes; copies of the insertion sequence of the transgenic corn GG2 were detected by southern blot. Results show that 1 band (FIG. 6) was hybridized, respectively from the enzyme-digested products of the HindIII and KpnI, which proves that the genes GAT and GR79 are inserted into the corn genome in one copy. Analysis results are shown in Table 3.

TABLE 3
Summary table of the southern blot results of the
genes of interest in the transgenic corn GG2

| | | | | Expected | | Actual | | |
|---|---|---|---|---|---|---|---|---|
| | | | | enzyme-digested | | enzyme-digested | | |
| | Probe | Target | Restriction | fragment band | | fragment band | | |
| Transformant | name | sequence | endonuclease | Number | Size/bp | Number | Size/bp | Copy |
| GG2 | GR79 | GR79 | HindIII | 1 | >5896 | 1 | ~8000 | 1 |
| | probe | gene | KpnI | 1 | >3841 | 1 | ~6500 | |
| | GAT | GAT | HindIII | 1 | >5896 | 1 | ~8000 | 1 |
| | probe | gene | KpnI | 1 | >2322 | 1 | ~6500 | |

### Example 8. Obtaining of a Left Border Flanking Sequence and a Right Border Flanking Sequence of the Transgenic Event GG2 by Chromosome Walking

Exogenous fragments inserted into the corn genome are shown in FIG. 7. Since the promoter ubiquitin is derived from a ubiquitin protein gene of corn, and the corn genome contains the gene, it is hard to obtain the right border flanking sequence by a chromosome walking method. Therefore, it began to design primers from GAT gene; the left border flanking sequence was amplified from the 5′ end to the 3′ end of the GAT gene.

1. Obtaining of the left border flanking sequence of the transgenic event GG2 by chromosome walking

Specific primers amplifying the left border flanking sequence were designed as follows:

GAT-SP1:
5′-GATGACGCACAATCCCAC-3′ (located on a CaMV 35S
promoter)
GAT-SP2:
5′-CTACGCTGGAGGGCTACA-3′ (located on the GAT gene)
GAT-SP4:
5′-GAGCAGGGCGAGGTGTTC-3′ (located on the GAT gene)

A Genome Walking Kit (Code No. 6108) was purchased from TaKaRa. The kit contains 4 types of degenerate primers; the 4 types of degenerate primers (AP1, AP2, AP3, and AP4) were amplified with GAT-SP1 and GAT-SP2 for two rounds, respectively. Based on the amplification effect, the primer AP4 was selected and amplified for the third time with GAT-SP4, thus obtaining a PCR product which was sent for sequencing.

(1) First round of PCR reaction

GAT-SP1 served as a forward primer, and 4 types of degenerate primers served as a reverse primer, respectively. AP4 was set as an example and subjected with the first round of PCR reaction.

Reaction system:

| | Use | |
|---|---|---|
| Component | amount | |
| Genomic DNA of GG2 | 100 | ng |
| dNTP Mixture (2.5 mM each) | 8 | μL |
| 10 × LA PCR BufferII (Mg²⁺ plus) | 5 | μL |
| TakaRa LA Taq (5 U/μL) | 0.5 | μL |
| AP4 Primer (100 pmol/μL) | 1 | μL |
| GAT-SP1 Primer (10 pmol/μL) | 1 | μL |

| | |
|---|---|
| ddH₂O | up to 50 μL |

Reaction conditions:

| | | | | | | | | | | |
|---|---|---|---|---|---|---|---|---|---|---|
| 94° C. | 1 | min | | | | | | | | |
| 98° C. | 1 | min | | | | | | | | |
| 94° C. | 30 | sec | | | | | | | | |
| 65° C. | 1 | min | {close oversize brace} | 5 | cycles | | | | | |
| 72° C. | 2 | min | | | | | | | | |
| 94° C. | 30 | sec | | 25° | C. | 3 min | 72° C. | 2 min | | |
| 94° C. | 30 | sec | | 65° | C. | 1 min | 72° C. | 2 min | | |
| 94° C. | 30 | sec | | 65° | C. | 1 min | 72° C. | 2 min | {close oversize brace} | 15 cycles |
| 94° C. | 30 | sec | | 44° | C. | 1 min | 72° C. | 2 min | | |
| 72° C. | 10 | min | | | | | | | | |

(2) Second round of PCR reaction 5 μL of the first round of PCR product was taken and subjected to electrophoresis (FIG. 8); the dilution ratio was selected according to the brightness of the first round of electrophoretic band. 1 μL of the diluted product of the first round of PCR reaction was taken as a template and subjected to the second first round of PCR; GAT-SP2 served as a forward primer, and 4 types of degenerate primers served as a reverse primer, respectively. AP4 was set as an example and subjected to the second round of PCR reaction.

Reaction system:

| | Use | |
|---|---|---|
| Component | amount | |
| First round of PCR reaction | 1 | μL |
| solution | | |
| dNTP Mixture (2.5 mM each) | 8 | μL |
| 10 × LA PCR BufferII (Mg²⁺ | 5 | μL |
| plus | | |
| TakaRa LA Taq (5 U/μL) | 0.5 | L |
| AP4 Primer (100 pmol/μL) | 1 | μL |
| GAT-SP2 Primer (10 pmol/μL) | 1 | μL |

| | |
|---|---|
| ddH₂O | up to 50 uL |

Reaction conditions:

| | | | | | | | |
|---|---|---|---|---|---|---|---|
| 94° C. | 30 sec | 65° C. | 1 min | 72° C. | 2 min | | |
| 94° C. | 30 sec | 65° C. | 1 min | 72° C. | 2 min | {close oversize brace} | 15 cycles |
| 94° C. | 30 sec | 44° C. | 1 min | 72° C. | 2 min | | |
| 72° C. | 10 min | | | | | | |

(3) Third round of PCR reaction 5 μL of the second round of PCR product was taken and subjected to electrophoresis (FIG. 8); the dilution ratio was selected according to the brightness of the second round of electrophoretic band. 1 μL of the diluted product of the second round of PCR reaction was taken as a template and subjected to the third round of PCR; GAT-SP4 served as a forward primer, and AP4 served as a reverse primer and subjected to the third round of PCR reaction.

Reaction system:

| | Use | |
|---|---|---|
| Component | amount | |
| Second round | 1 | μL |
| of PCR reaction | | |
| solution | | |
| dNTP Mixture | 8 | μL |
| (2.5 mM each) | | |
| 10 × LA PCR | 5 | μL |
| BufferII (Mg²⁺ plus) | | |
| TakaRa LA Taq | 0.5 | μL |
| (5 U/μL) | | |
| AP4 Primer | 1 | μL |
| (100 pmol/μL) | | |
| GAT-SP4 Primer | 1 | μL |
| (10 pmol/μL) | | |

| | |
|---|---|
| ddH₂O | up to 50 μL |

Reaction conditions:

| | | | | | | | |
|---|---|---|---|---|---|---|---|
| 94° C. | 30 sec | 65° C. | 1 min | 72° C. | 2 min | | |
| 94° C. | 30 sec | 65° C. | 1 min | 72° C. | 2 min | {close oversize brace} | 15 cycles |
| 94° C. | 30 sec | 44° C. | 1 min | 72° C. | 2 min | | |
| 72° C. | 10 min | | | | | | |

(4) 5 μL of the third round of PCR product was taken and subjected to electrophoresis with 1% agarose gel; the electrophoretogram is shown in FIG. 8. The gel was cut and a clear electrophoretic band was recovered. The third round of the PCR product was subjected to DNA sequencing with GAT-SP4 as primers.

Sequencing results are shown in SEQ ID NO. 6. By sequence alignment, the T-DNA sequence of GG2 is located at positions 1-268 of SEQ ID NO.6; 22 bp is missing at the 3′ end of the left border sequence of the vector. The sequence of 269-462 of SEQ ID NO.6 is located at chr1:269325682-269325493 (Zea mays (B73_RefGen_v4).

The sequencing result of the flanking sequence at 3′ end of the exogenous insertion fragment of the transgenic glyphosate-resistant corn GG2 by chromosome walking:

| | |
|---|---|
| 1 | CGCGTGACTC GAGTTTCTCC ATAATAATGT GTGAGTAGTT CCCAGATAAG GGAATTAGGG |
| 61 | TTCCTATAGG GTTTCGCTCA TGTGTTGAGC ATATAAGAAA CCCTTAGTAT GTATTTGTAT |
| 121 | TTGTAAAATA CTTCTATCAA TAAAATTTCT AATTCCTAAA ACCAAAATCC AGTACTAAAA |
| 181 | TCCAGATCCC CCGAATTAAT TCGGCGTTAA TTCAGTACAT TAAAAACGTC CGCAATGTGT |
| 241 | TATTAAGTTG TCTAAGCGTC AATTTGTTAT TCTTATCATC ATGTAAAATA CGTACAACAC |
| 301 | ATTGCATGAC TCGTCATGCA CGCACTTGGG CTGCTGCTGC TTTACATGCA CGCGCGCACC |
| 361 | GACGGCCGGC CGGTGGTGCT GATCAGAAAT GTACACGCCT GTGAGGCAGG CAGGCAGAGA |
| 421 | GAGAGAGAGA GAGATACATA TTCACACACG CACGCACGCA CC |

The underline denotes the corn genomic sequence.

2. Obtaining of the right border flanking sequence of the insertion fragment

Based on the obtained left border flanking sequence, the known corn genomic sequences were searched; specific primers were designed on the ubiquitin promoter sequence of the vector and the speculated right border flanking sequence and subjected to PCR amplification.

Specific primers:

GG2-Ubi-F1: 5′-ATGATTCTCTAAAACACTG-3′ (located on the Ubiquitin promoter sequence)

GG2-Right-R1: 5′-GCGAACATAGCGTCTTAC-3′ (located on the corn genome)

Size of the PCR product: 1773 bp.

Reaction system:

| | Use | |
|---|---|---|
| Component | amount | |
| Genomic DNA of GG2 | 100 | ng |
| GG2-Ubi-F1 (10 pmol/μL) | 0.5 | μL |
| GG2-Right-R1 (10 pmol/μL) | 0.5 | μL |
| 2 × Taq Plus Master | 10 | μL |
| Mix II (Dye Plus) | | |

| | |
|---|---|
| ddH₂O | up to 20 μL |

Reaction conditions:

| 95° C. | 3 min | | |
|---|---|---|---|
| 95° C. | 15 sec | | |
| 50° C. | 20 sec | | 30 cycles |
| 72° C. | 1 min | {close oversize brace} | |
| | 20 sec | | |

| | |
|---|---|
| 72° C. | 5 min |

2 μL of the PCR product was taken and subjected to electrophoresis detection. Results are shown in FIG. 9. The remaining PCR product was subjected to DNA sequencing.

Sequencing results are shown in SEQ ID NO.7. By sequence alignment, the T-DNA sequence of the vector's right border is located at positions 1-435 of SEQ ID NO.7; 46 bp is missing at the 5′ end of the right border sequence of the vector. A recombined sequence is located at positions 436-455. The sequence of 456-1773 of SEQ ID NO.7 is located at chr1:269325753-269326914 (Zea mays (B73_RefGen_v4).

The specificity PCR sequencing result of the flanking sequence at 5′ end of the exogenous insertion fragment of the transgenic glyphosate-resistant corn GG2.

| | |
|---|---|
| 1 | ATGATTCTCT AAAACACTGA TATTATTGTA GTACTATAGA TTATATTATT CGTAGAGTAA |
| 61 | AGTTTAAATA TATGTATAAA GATAGATAAA CTGCACTTCA AACAAGTGTG ACAAAAAAAA |
| 121 | TATGTGGTAA TTTTTTATAA CTTAGACATG CAATGCTCAT TATCTCTAGA GAGGGGCACG |
| 181 | ACCGGGTCAC GCTGCACTGC AGGCATGCAA GCTTGGCACT GGCCGTCGTT TTACAACGTC |
| 241 | GTGACTGGGA AAACCCTGGC GTTACCCAAC TTAATCGCCT TGCAGCACAT CCCCCTTTCG |
| 301 | CCAGCTGGCG TAATAGCGAA GAGGCCCGCA CCGATCGCCC TTCCCAACAG TTGCGCAGCC |
| 361 | TGAATGGCGA ATGCTAGAGC AGCTTGAGCT TGGATCAGAT TGTCGTTTCC CGCCTTCAGT |
| 421 | TTAAACTATC AGTGTGACCG TGGAACACGA ATCGCCGAAT CGGTGCGTGC GTGAGTGCGT |
| 481 | GCTTCATCGA TTCAGACTAT CGGGCGTGTT CGGCTGGTTG CAAGCCGACA CTGTTGCAGC |
| 541 | TGTTTGGACT GCTGCAGCTG CAATCCATAG AGAGAAAAAT ACTGTAGAAG CCGCAGCCGC |
| 601 | AGCCGGATTG CAGCCGCAGC AAGCCGCAGC GAACAAGCTG ATCGTCTACG CTGGGTACGT |
| 661 | GCTGGCGACT CAAATCATCG ATTGGACGGT GGCGTCGCGC GGCGGGGAGG CGTTGCCGTT |
| 721 | GGGCGATGAC GCGGACGGAT GCAGATGGAA TGACGTCGCG TCGTTGCGTG CGTTCTACTT |
| 781 | CTATAAACTA CGCCATCGAT CTCCTGCCTG GCTGGCATCG GTCGCCACGC ACGCATGTTT |
| 841 | GTCTATTCTC CGTCCGTCAC GTCACCCTCG CGCTGTCGTC GCTATGCACA CGGTCGGCCT |
| 901 | CACCCTCGCG GATCACGTCG CAGTCGCCGT CGCCGTCGCC GTCGCCACCA AGTCATCGTC |
| 961 | GCGTCGGCGC GACAGCGAGG GCGCACGCGC GCCACGCTAA GCTCAGACGA GGGACACGAC |
| 1021 | GACCTGGCCT TATCGGCTCT GATAGCGTGC CTAGCCGGAT CGGAGAGGGG CGCAGTGGCC |
| 1081 | AGTGTGGGGG GTGTCACTGT CAGTCACGGT TCTTGTCCGC CCGATCGCAT CCCGATAGCC |
| 1141 | TTCTGCTCGG AGCCTCTGTC CGCCTGTCTG TCGTGTGCCT GTAAAATCAG TGTGGGTTGG |
| 1201 | AGTGCGCGCG CGCGTTTCTG ATGGCTGATG CTGCCATCGT TAAAATCGGT GTGGGAGATG |
| 1261 | TTGAGCTTGA GCGTGCACAT GCATGTCCAG GTCCACCATG TATTTATTTG CCATGCTGCG |
| 1321 | ATGATGGCCC TTTAGGCAAA CAGGTTCTAG AGCAGTTCGA TTCTGTGTAT AACCGAGCCG |
| 1381 | CTTAAATTTT TTTTAAAAAA TATTTCTCGT AGCTGCTGCA TCTGGCAGCT TCCAAAAATA |
| 1441 | AGGATATTCT TTATAGATGT TGCATCTATT CTAGGGGCTC CCTACAATCG ACAGAGCCTA |
| 1501 | ATGACACGCT ACCGGCTACT CCCTCGTCCC ATTCAGAGCT TGTTCGGTTA TTTACAATCC |
| 1561 | ATATGGATTG GAGGGGATTG ATACGGATTG GAGAGAATTT TGACTTACTA GGGATTGAAA |
| 1621 | CCCCCTCAAT CCATATGGAT TGAGGTAGAA CCGAACAAGC CCTCAGGACA TGTTCGGTTA |
| 1681 | CACCAATCCA GAAGGGGATT ACAATCCAGA AGGGTCAATC CCCTTCTGGA TTGGTGTAAC |
| 1741 | CGAACAAGCC CTCACGTAAG ACGCTATGTT CGC |

The underline denotes the corn genomic sequence.

3. Obtaining of the left border flanking sequence of the insertion fragment Since the left border flanking sequence obtained by chromosome walking was too short (less than 300 bp), to obtain a longer left border flanking sequence, the known corn genomic sequences were searched; specific primers were designed on the GAT gene sequence of the vector and the reference sequence of the corn left border genome and subjected to PCR amplification.

Specific primers:
GG2-Left-F3:
5′-GGAGCAAGGAAGCGGACTAC-3′ (located on the corn
genome)
GG2-Left-R1:
5′-CCCCACATCCTGATGTACAAG-3′ (located on the gat
gene sequence)

Size of the PCR product: 734 bp.

Reaction system:

| | Use | |
|---|---|---|
| Component | amount | |
| Genomic DNA of GG2 | 100 | ng |
| GG2-Left-F3 (10 pmol/μL) | 0.5 | μL |
| GG2-Left-R1 (10 pmol/μuL) | 0.5 | μL |
| 2 × Taq Plus Master | 10 | μL |
| Mix II (Dye Plus) | | |

| | |
|---|---|
| ddH₂O | up to 20 μL |

Reaction conditions:

| 95° C. | 3 min | | |
|---|---|---|---|
| 95° C. | 15 sec | | |
| 58° C. | 20 sec | {close oversize brace} | 30 cycles |
| 72° C. | 50 sec | | |

| | |
|---|---|
| 72° C. | 5 min |

2 μL of the PCR product was taken and subjected to electrophoresis detection. Results are shown in FIG. 9. The remaining PCR product was subjected to DNA sequencing.

In combination with the sequencing results of the left and right border flanking sequences, the position of the insertion fragment in corn is shown in FIG. 10. The T-DNA sequence of the transgenic corn GG2 was integrated into positions 269325682-269325753 (Zea mays (B73_RefGen_v4)) of Chr1 of the corn genome, leading to the deletion of a 70 bp sequence at the insertion sites of the corn genome, corresponding to chr1: 269325683-269325752 (Zea mays (B73_RefGen_v4)). The exogenous insertion sequence of the transgenic corn GG2 is shown at positions 441-6535 of SEQ ID NO.8; the left border flanking sequence is shown at positions 1-440 of SEQ ID NO.8, and the right border flanking sequence is shown at positions 6556-7873 of SEQ ID NO.8.

The transgenic corn GG2 is a transgenic corn event with high glyphosate herbicide tolerance and has important values of production and application. The flanking sequences at both ends of the exogenous insertion fragment of the transgenic glyphosate-resistant corn GG2 and specific primers thereof may serve as molecular markers, so to as detect the transgenic glyphosate-resistant corn event GG2 and its derived materials.

### Example 9. Detection of Tolerance of the Bivalent GAT and GR79

transgenic corn to non-target herbicide 3 types of herbicides were set in this test, i.e., mesotrione· propisochlor atrazine SC (3.5% mesotrione, 15% propisochlor, and 15% atrazine) commonly used in corn field, glufosinate-ammonium (20% active ingredient) sensitive to corn, and haloxyfop-R-methyl (10.8% active ingredient), respectively. 4 treatments were set: (1) free of spraying herbicide; (2) spraying mesotrione· propisochlor atrazine SC; (3) spraying glufosinate-ammonium; and (4) spraying haloxyfop-R-methyl. The spraying dose is a high dose on the pesticide registration tag. Stems and leaves were sprayed when corn grew to four to six leaves. Seedling rate and symptom of pesticide damage were surveyed 1, 2, and 4 weeks later after application, respectively. The results indicate that: in the treatments of spraying the same dose of non-target herbicide mesotrione. propisochlor atrazine SC, both the bivalent GAT and GR79 transgenic herbicide-resistant corn and the non-transgenic control corn were free of symptom of herbicide damage and the plants grow well; in the treatments of spraying the same dose of glufosinate-ammonium and haloxyfop-R-methyl, respectively, all the bivalent GAT and GR79 transgenic herbicide-resistant corn and the non-transgenic control corn died (FIG. 11).

Mesotrione·propisochlor·atrazine SC is a kind of corn post-emergence herbicide, and causes no harm to the transgenic corn and the non-transgenic corn. Glufosinate-ammonium and haloxyfop-R-methyl are seriously harmful to corn. From the angle of safety of transgenic organism, the glyphosate-resistant corn is sensitive to glufosinate-ammonium and haloxyfop-R-methyl, indicating that the glyphosate-tolerant corn may be killed by other herbicides, thus preventing its excessive growth and preventing it from growing into a “field superweed”.

1. An expression vector, comprising two glyphosate-resistant genes GR79 and GAT, wherein the GR79 gene has a nucleotide sequence as shown in SEQ ID NO.3, and the GAT gene has a nucleotide sequence as shown in SEQ ID NO.4.

2. The expression vector according to claim 1, named a plant expression vector pCGG and having a skeleton vector of pCAMBIA2300.

3. The expression vector according to claim 2, wherein the plant expression vector pCGG has a structure as shown in FIG. 2.

4. The expression vector according to claim 3, wherein the plant expression vector pCGG has a nucleotide sequence as shown in SEQ ID NO.5.

5. Use of the expression vector according to any one of claims 1-4 in glyphosate tolerance of a plant, wherein the use is to transform the expression vector according to any one of claims 1-4 into a plant to express a GR79 protein and a GAT protein such that the plant has a character of glyphosate resistance.

6. The use according to claim 5, wherein a method for the transformation is an Agrobacterium-mediated transformation method.

7. The use according to claim 5, wherein the plant is a corn.

8. A glyphosate-resistant corn transformed with genes GR79 and GAT, wherein the GR79 gene has a nucleotide sequence as shown in SEQ ID NO.3, and the GAT gene has a nucleotide sequence as shown in SEQ ID NO.4.

9. A left border flanking sequence of an exogenous insertion fragment of the glyphosate-resistant corn transformed with genes GR79 and GAT according to claim 8, as shown in positions 1-440 of SEQ ID NO.8.

10. A right border flanking sequence of an exogenous insertion fragment of the glyphosate-resistant corn transformed with genes GR79 and GAT according to claim 8, as shown in positions 6556-7873 of SEQ ID NO.8.

11. A specific primer pair for PCR reaction detection designed according to the left border flanking sequence of claim 9.

12. The specific primer pair for PCR reaction detection designed according to the left border flanking sequence of claim 11, comprising a sequence:
GG2-Left-F3:
5′-GGAGCAAGGAAGCGGACTAC-3′,
GG2-Left-R1:
5′-CCCCACATCCTGATGTACAAG-3′.

13. A specific primer pair for PCR reaction detection designed according to the right border flanking sequence of claim 10.

14. The specific primer pair for PCR reaction detection designed according to the right border flanking sequence of claim 13, comprising a sequence:
GG2-Ubi-F1:
5′-ATGATTCTCTAAAACACTG-3′,
GG2-Right-R1:
5′-GCGAACATAGCGTCTTAC-3′.

15. A PCR reaction detection method of a transgenic glyphosate-resistant corn GG2, wherein a primer pair in the PCR reaction is the specific primer pair according to any one of claims 11-14.

16. The PCR reaction detection method according to claim 15, wherein the specific primer pair is:
GG2-Left-F3:
5′-GGAGCAAGGAAGCGGACTAC-3′,
GG2-Left-R1:
5′-CCCCACATCCTGATGTACAAG-3′,
a fragment obtained by the PCR reaction has a size of 734 bp; or
the specific primer pair is:
GG2-Ubi-F1:
5′-ATGATTCTCTAAAACACTG-3′,
GG2-Right-R1:
5′-GCGAACATAGCGTCTTAC-3′.
a fragment obtained by the PCR reaction has a size of 1773 bp.

17. A kit for detecting a glyphosate-resistant corn, comprising the specific primer pair for PCR reaction detection designed according to the left border flanking sequence of claim 11 or 12, and/or the specific primer pair for PCR reaction detection designed according to the right border flanking sequence of claim 13 or 14.

18. Use of the flanking sequence according to claim 9 or 10, the specific primer pair according to any one of claims 11-14, and the kit for detecting a glyphosate-resistant corn according to claim 17 in detecting a transgenic corn.
